(19) United States
(12) Patent Application Publication
(10) Pub. No.: US 2010/0305440 A1
(43) Pub. Date: Dec. 2, 2010

(54) Ultrasound System And Method For Providing A Motion Vector

(75) Inventors: Hyeong Do LEE, Seoul (KR); Jeong Sik Kim, Seoul (KR)

Correspondence Address: JONES DAY, 222 EAST 41ST ST, NEW YORK, NY, 10017, US

(73) Assignee: Medison Co., Ltd.

(21) Appl. No.: 12/790,567

(22) Filed: May 28, 2010

(30) Foreign Application Priority Data
Jun. 1, 2009 (KR) 10-2009-0048206
May 6, 2010 (KR) 10-2010-0042290

(51) Int. Cl. A61B 8/00

(52) U.S. Cl. 600/441

(57) ABSTRACT

A method of providing a motion vector is disclosed. The method according to embodiments of the present disclosure comprises: acquiring ultrasound data corresponding to a region of interest within a brightness mode (B-mode) image of a target object; forming a color Doppler mode image of the target object based on the ultrasound data; forming a motion vector of the target object based on the ultrasound data and the color Doppler mode image; and setting the motion vector on the color Doppler mode image.

### CROSS-REFERENCE TO RELATED APPLICATIONS

The present application claims priority from Korean Patent Application Nos. 10-2009-0048206, filed on Jun. 1, 2009, and 10-2010-0042290, filed on May 6, 2010, the entire subject matters of which are incorporated herein by reference.

### TECHNICAL FIELD

The present disclosure relates to ultrasound systems, and more particularly to an ultrasound system and method of providing a motion vector, which comprises the speed and direction of movement of a moving object of interest.

### BACKGROUND

Due to its non-invasive and non-destructive nature, an ultrasound system has been extensively used in the medical field to acquire internal information of a target object. The ultrasound system is used in the medical field since it can provide doctors with a high resolution image of internal tissues of the target object without the need of surgical treatment.

As is known in the art, the ultrasound system may function in various modes including a brightness mode (B-mode) wherein reflection coefficients of the ultrasound signal reflected by the target object may be visualized, a Doppler mode (D-mode) wherein speed information of a moving target object (especially bloodstream) may be acquired by using the Doppler effect, and an elastic mode (E-mode) wherein mechanical characteristics of tissues may be visualized based on strains representing deformation of the tissues due to the application of the stress.

The Doppler mode is based on a Doppler frequency, which is the difference between the frequency of the transmitted ultrasound signal from an ultrasound probe (“transmission frequency”) and the frequency of the echo signal reflected from the moving target object and received through the ultrasound probe (“reception frequency”). The Doppler frequency varies according to the angle (“Doppler angle”) between an ultrasound beam consisting of a group of ultrasound signals and the reflecting object (i.e., bloodstream). For example, assuming that the reflecting object is moving at a speed of 1 m/s and the transmission frequency is 5 MHz, the reception frequency would be 6.5 MHz when the Doppler angle is 0° and 0 MHz when the Doppler angle is 90°. As such, the speed of the bloodstream is difficult to estimate if the Doppler angle is 90°. Further, for a convex probe, the bloodstream may be displayed as moving in the direction opposite to its actual direction depending on the angle of the ultrasound beam.

To solve the above problems, a method of using two ultrasound beams or speckle information has been proposed to detect the speed of the bloodstream having the Doppler angle of 90°. However, such a method is not practical since the performance is not stable or the frame rate is too low to allow for real time display.

### SUMMARY

According to the present disclosure, there is disclosed an ultrasound system and method for providing a motion vector of a moving object of interest (e.g., bloodstream) by using a color Doppler mode image and ultrasound data comprising speed information of the object of interest.

According to an aspect of the present disclosure, a method of providing a motion vector on an ultrasound image comprises: acquiring a brightness mode (B-mode) image of a target object; enabling a region of interest (ROI) to be set in the B-mode image, the ROI covering a moving object in the target object; acquiring ultrasound data for a portion of the target object corresponding to the ROI, the ultrasound data including speed information of the moving object; forming a color Doppler mode image of the portion based on the ultrasound data; forming a motion vector of the moving object based on the ultrasound data and the color Doppler mode image; and setting the motion vector on the color Doppler mode image.

According to another aspect of the present disclosure, a system for providing a motion vector on an ultrasound image comprises: an image acquisition unit configured to acquire a brightness mode (B-mode) image of a target object; a region setting unit configured to enable a region of interest (ROI) to be set in the B-mode image, the ROI covering a moving object in the target object; an ultrasound data acquisition unit configured to acquire ultrasound data for a portion of the target object corresponding to the ROI, the ultrasound data including speed information of the moving object; an image forming unit configured to form a color Doppler mode image of the portion based on the ultrasound data; a processor configured to form a motion vector of the moving object based on the ultrasound data and the color Doppler mode image and to set the motion vector on the color Doppler mode image.

The ultrasound system and method according to the present disclosure can provide a motion vector comprising a speed and direction of movement of a moving object of interest (e.g., bloodstream) without lowering the frame rate. Thus, it can accurately provide a user with the direction of movement of the moving object of interest.

### BRIEF DESCRIPTION OF THE DRAWINGS

FIG. 1 illustrates a block diagram showing an arrangement of an ultrasound system according to an embodiment of the present disclosure.

FIG. 2 illustrates a block diagram showing an arrangement of an ultrasound data acquisition unit according to an embodiment of the present disclosure.

FIG. 3 illustrates a schematic diagram showing transducer elements, scanlines and coordinate system according to an embodiment of the present disclosure.

FIG. 4 illustrates a block diagram showing an arrangement of a processor according to an embodiment of the present disclosure.

FIG. 5 illustrates a diagram showing a B-mode image, a Doppler mode image, a region of interest and a bloodstream according to an embodiment of the present disclosure.

FIG. 6 illustrates a diagram showing lines and line intersections according to an embodiment of the present disclosure.

FIG. 7 illustrates a diagram showing an area including 5×5 pixels according to an embodiment of the present disclosure.

FIG. 8 illustrates a diagram showing a motion vector formed according to an embodiment of the present disclosure.

FIG. 9a illustrates an enlarged view of portion A shown in FIG. 8.

FIG. 9b illustrates an enlarged view of portion B shown in FIG. 8.

### DETAILED DESCRIPTION

Embodiments of the present disclosure are described below with reference to the accompanying drawings. As used herein, the term “Doppler mode” comprises a color Doppler mode. In addition, a target object comprises a moving object of interest (e.g., bloodstream).

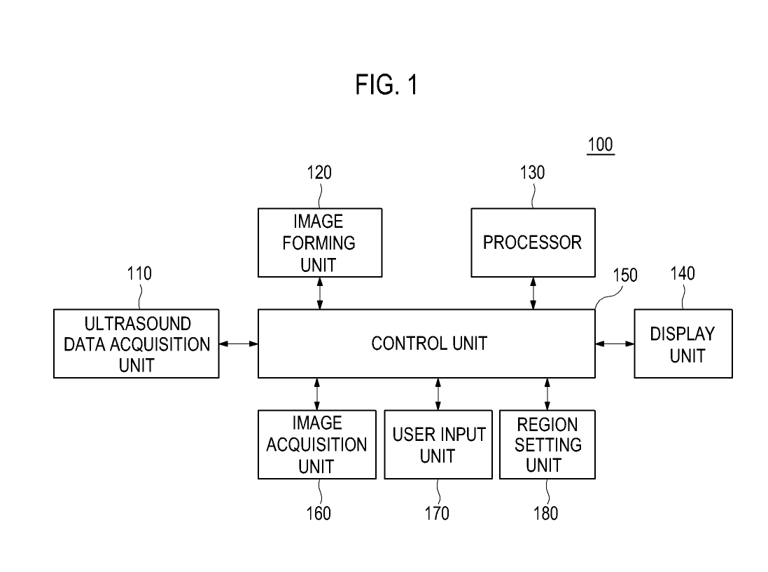

FIG. 1 illustrates a block diagram showing an arrangement of an ultrasound system 100 according to an embodiment of the present disclosure and that embodies the methods of the present invention. The ultrasound system 100 may comprise an ultrasound data acquisition unit 110, an image forming unit 120, a processor 130, a display unit 140 and a control unit 150. The ultrasound system 100 may further comprise an image acquisition unit 160, which is configured to acquire a brightness mode (B-mode) image of a target object, a user input unit 170, which is configured to receive, from a user, information on a region of interest (ROI) (i.e., color box) for acquiring a Doppler mode image of the object of interest, and a region setting unit 180, which is configured to enable the ROI to be set in the B-mode image, the ROI covering a moving object in the target object. The user input unit 170 may comprise a control panel, a mouse, a keyboard and the like. In this embodiment, the information on the region of interest may include the size and location of the region of interest within the B-mode image.

The ultrasound data acquisition unit 110 may transmit an ultrasound signal to a target object and receive the ultrasound signal reflected by the target object (i.e., ultrasound echo signal), thereby acquiring ultrasound data corresponding to scanlines of the region of interest within the B-mode image.

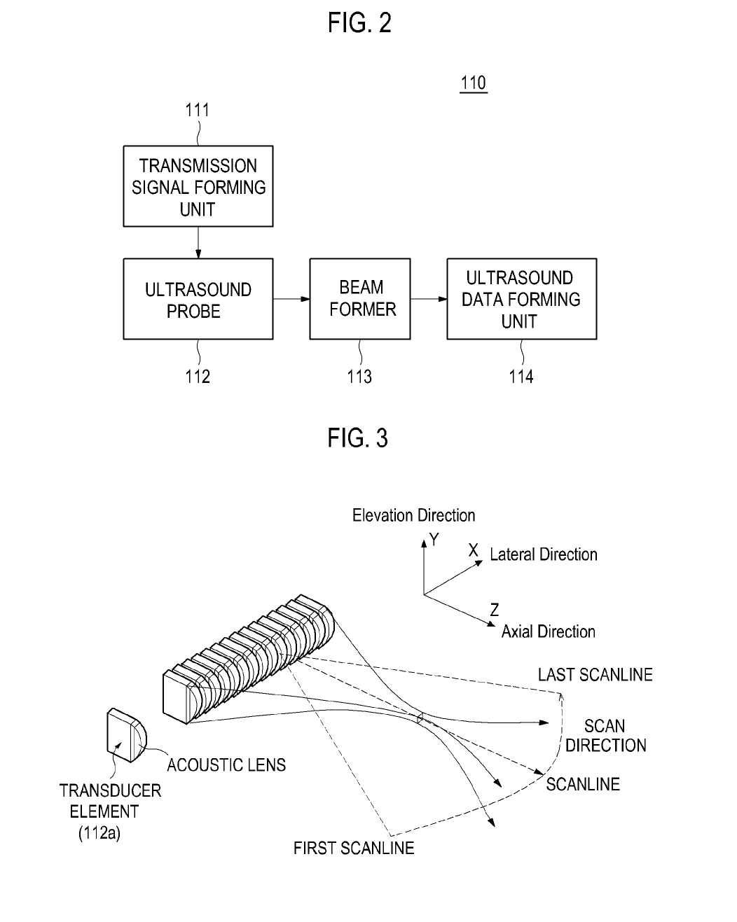

FIG. 2 illustrates a block diagram showing an arrangement of the ultrasound data acquisition unit 110 according to an embodiment of the present disclosure. The ultrasound data acquisition unit 110 may comprise a transmission signal forming unit 111, an ultrasound probe 112 including multiple transducer elements, a beam former 113 and an ultrasound data forming unit 114.

The transmission signal forming unit 111 may form transmission signals, which will be applied to the ultrasound probe 112 for acquiring a Doppler mode image in consideration of the focal points and locations of the transducer elements of the ultrasound probe 112.

The ultrasound probe 112 may convert the transmission signals provided from the transmission signal forming unit 111 into ultrasound signals, transmit the ultrasound signals to the target object, and receive the ultrasound echo signals reflected by the target object to thereby form reception signals. The ultrasound probe 112 may comprise multiple transducer elements 112a configured to perform inter-conversion between an ultrasound signal and an electrical signal, as shown in FIG. 3. A group of ultrasound signals transmitted from the multiple transducer elements 112a are formed into ultrasound beams, and then transmitted to the target object along the respective multiple scanlines, as shown in FIG. 3. FIG. 3 shows the lateral direction perpendicular to the axial direction and the elevation direction which is the thickness direction in cross section of the target object.

The beam former 113 may perform analog-to-digital conversion of the reception signals provided by the ultrasound probe 112. The beam former 113 may form receive-focused signals by receive-focusing the digital-converted reception signals in consideration of the focal points and locations of the transducer elements of the ultrasound probe 112.

The ultrasound data forming unit 114 may form ultrasound data corresponding to each of the multiple scanlines using the receive-focused signals provided by the beam former 113. The ultrasound data may comprise speed information of the object of interest. The ultrasound data forming unit 114 may further perform multiple signal processing (e.g., gain control, filtering, etc.) to form a Doppler mode image.

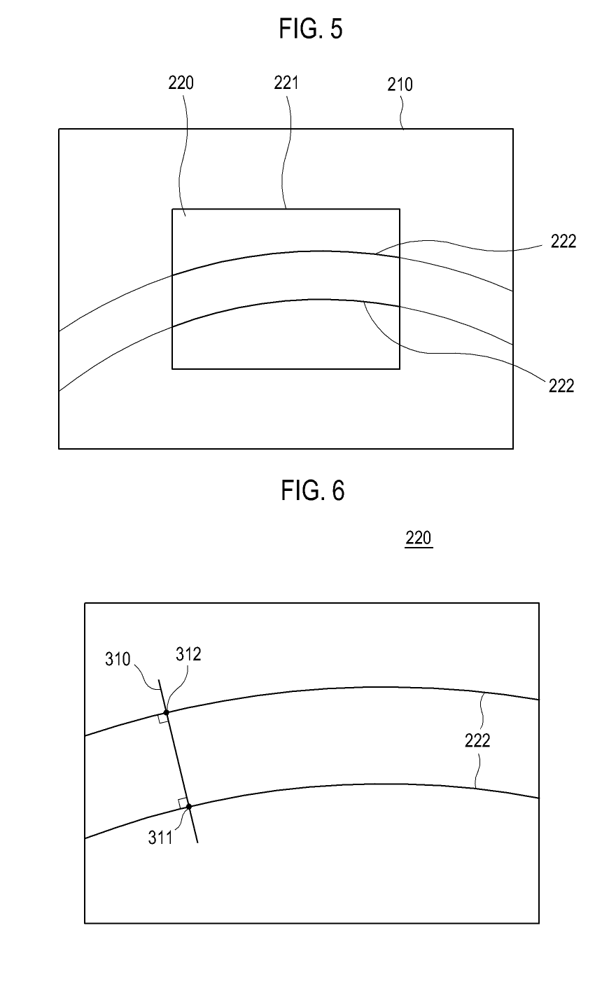

Referring back to FIG. 1, the image forming unit 120 may form a Doppler mode image 220, in which the speed of the object of interest (e.g., bloodstream) approaching the ultrasound probe 112 and the speed of the object of interest receding from the ultrasound probe 112 are represented in different colors. In FIG. 5, reference numerals 210, 221 and 222 represent the B-mode image, the region of interest and the blood vessel, respectively.

The processor 130 may set a motion vector, which comprises the speed and direction of movement of the object of interest, by using the ultrasound data provided by the ultrasound data acquisition unit 110 and the Doppler mode image provided by the image forming unit 120.

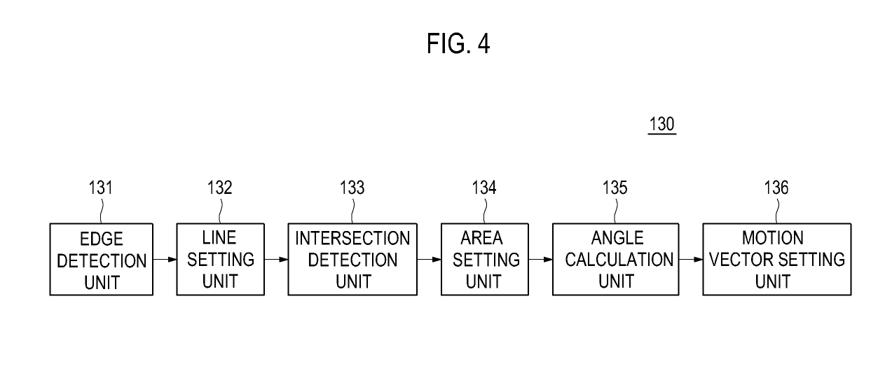

FIG. 4 illustrates a block diagram showing an arrangement of a processor 130 according to an embodiment of the present disclosure. The processor 130 may comprise an edge detection unit 131, a line setting unit 132, an intersection detection unit 133, an area setting unit 134, an angle calculation unit 135 and a motion vector setting unit 136.

The edge detection unit 131 may detect an edge (e.g., the edge of blood vessel 222) by analyzing the Doppler mode image 220 provided by the image forming unit 120. The edge may be detected based on the brightness changes and using differential operators. In this embodiment, the edge may be detected by using an edge mask such as Sobel, Prewitt, Robert, Canny mask, etc. Alternatively, the edge may be detected based on the differences between eigenvalues and using structure tensors.

The line setting unit 132 may set lines perpendicular to the edge (e.g., the edge of blood vessel 222) detected by the edge detection unit 131. As an example, the line setting unit 132 may set a line 310, which is perpendicular to the edge (e.g., the edge of blood vessel 222), as described in FIG. 6.

The intersection detection unit 133 may detect first and second intersections 311 and 312, each of which is the intersection of the edge (e.g., the edge of blood vessel 222) and the line set by the line setting unit 132.

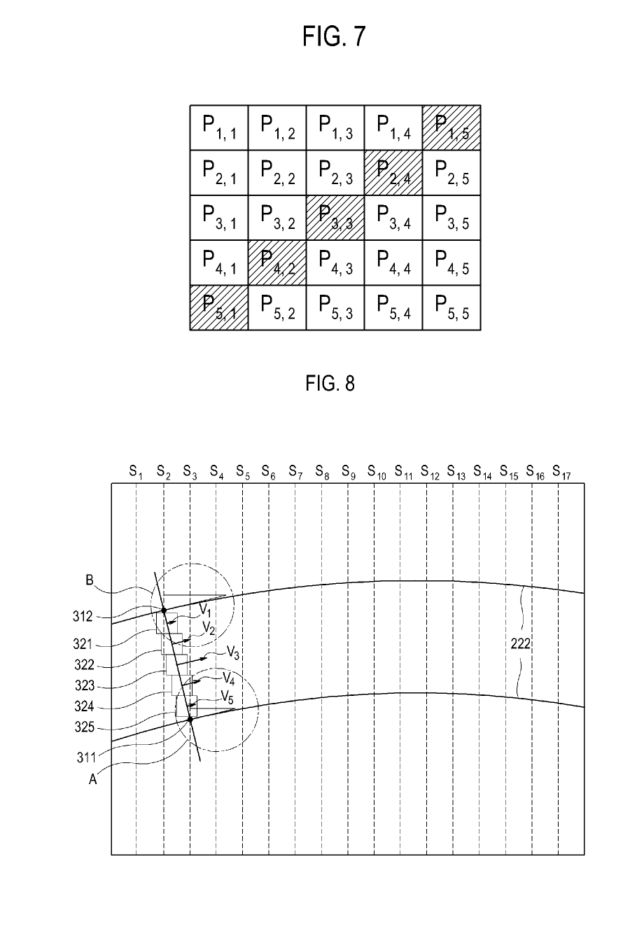

The area setting unit 134 may set first areas including a pre-defined number of pixels, wherein each of the first areas is centered at the intersections detected by the intersection detection unit 133. As an example, the area setting unit 134 may set a first area, which includes 5×5 pixels and is centered at the first intersection P_{3, 3} 311 as described in FIG. 7. In FIG. 7, shaded pixels P_{1, 5}, P_{2, 4}, P_{3, 3}, P_{4, 2}, P_{5, 1} represent the edge. The area setting unit 134 may also set a first area for the second intersection 312, which includes 5×5 pixels and is centered at the second intersection 312 as described above.

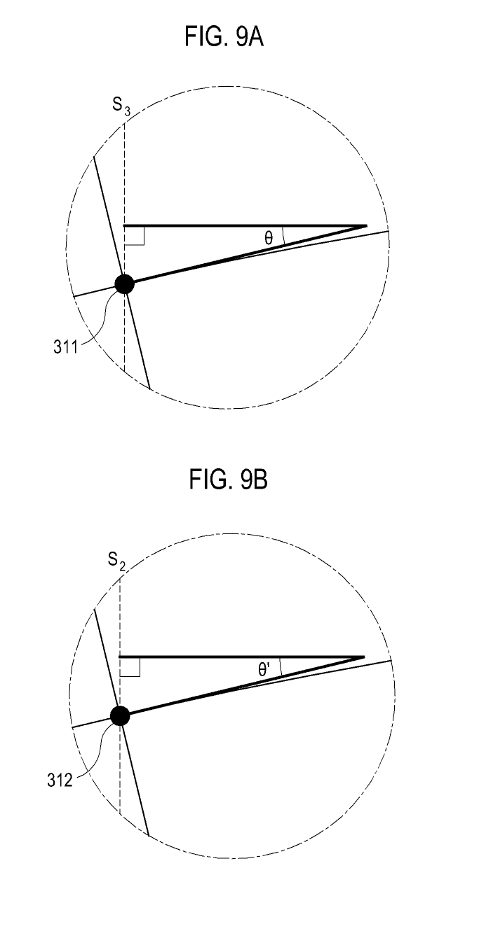

The angle calculation unit 135 may detect pixels corresponding to the edge for each of the first and second intersections 311 and 312, and calculate angles of the edges at each of the first and second intersections 311 and 312 by using the detected pixels. As an example, the angle calculation unit 135 may detect pixels P_{1, 5}, P_{2, 4}, P_{3, 3}, P_{4, 2}, P_{5, 1} corresponding to the edge for the first intersection 311 within the area including 5×5 pixels, as shown in FIG. 7. The angle calculation unit 135 may calculate the angle θ of the edge at the first intersection 311 by using the left-most pixel P_{5, 1} and the right-most pixel P_{1, 5} among the detected pixels, as shown in FIG. 9a. The methods of calculating the angle of the edge are well known in the art. Thus, they have not been described in detail so as not to unnecessarily obscure the present disclosure. Similarly, the angle calculation unit 135 may also calculate the angle θ′ for the second intersection 312, as shown in FIG. 9b.

The motion vector setting unit 136 may form a motion vector, which includes a speed and direction of movement of the object of interest, by using ultrasound data provided by the ultrasound data acquisition unit 110 and the angle calculated by the angle calculation unit 135. The motion vector setting unit 136 may then set the motion vector on the Doppler mode image.

In particular, the motion vector setting unit 136 may set a plurality of second areas 321-325 on the line segment demarcated by the first intersection 311 and the second intersection 312 on the line 310, as shown in FIG. 8. Each of the second areas 321-325 may have a predetermined size (e.g., 5×5), and the center of each of the second areas 321-325 may be located on the line 310. In one embodiment, the second areas 321-325 may be set in an overlapping manner. Although the foregoing embodiment has been described to set the five second areas 321-325 on the line 310, the present disclosure is not limited thereto.

In one embodiment, the motion vector setting unit 136 may obtain object-of-interest information corresponding to each of the second areas 321-325 by using the ultrasound data for the scanline corresponding to the respective second area. The object-of-interest information comprises the size (speed) and direction of movement of the moving object of interest (e.g., bloodstream). In another embodiment, the motion vector setting unit 136 may obtain object-of-interest information by using the ultrasound data for the scanline most adjacent the respective second area.

The motion vector setting unit 136 may calculate an average speed in each of the second areas 321-325 by using the respectively obtained object-of-interest information. The motion vector setting unit 136 may form motion vectors V₁-V₅ for the second areas 321-325 based on the calculated average speeds and the angle of the edge calculated by the angle calculation unit 135, and may set the formed motion vectors V₁-V₅ on the Doppler mode image 220.

Referring back to FIG. 1, the display unit 140 may display the Doppler mode image 220 and the motion vectors V₁-V₅.

The control unit 150 may control the transmission and receiving of the ultrasound signals, and control the formation of the ultrasound data. The control unit 150 may also control the formation and display of the Doppler mode image, and control the formation and display of the vectors.

In another embodiment, the present invention may provide a method of providing a motion vector on an ultrasound image comprises: acquiring a brightness mode (B-mode) image of a target object; enabling a region of interest (ROI) to be set in the B-mode image, the ROI covering a moving object in the target object; acquiring ultrasound data for a portion of the target object corresponding to the ROI, the ultrasound data including speed information of the moving object; forming a color Doppler mode image of the portion based on the ultrasound data; forming a motion vector of the moving object based on the ultrasound data and the color Doppler mode image; and setting the motion vector on the color Doppler mode image.

While the present disclosure is described via some preferred embodiments, it will be appreciated by those skilled persons in the art that many modifications and changes can be made without departing the spirit and scope of the appended claims.

As an example, while only one line 310 perpendicular to the edge (e.g., the edge of blood vessel 222) and second areas on the line 310 are used to form a motion vector of the object of interest in the foregoing embodiments, at least two different lines perpendicular to the edge (e.g., the edge of blood vessel 222) at different locations and second areas on the at least two different lines may be used to form the motion vector of the object of interest in other embodiments.

What is claimed is:

1. A method of providing a motion vector on an ultrasound image, comprising:
acquiring a brightness mode (B-mode) image of a target object;
enabling a region of interest (ROI) to be set in the B-mode image, the ROI covering a moving object in the target object;
acquiring ultrasound data for a portion of the target object corresponding to the ROI, the ultrasound data including speed information of the moving object;
forming a color Doppler mode image of the portion based on the ultrasound data;
forming a motion vector of the moving object based on the ultrasound data and the color Doppler mode image; and
setting the motion vector on the color Doppler mode image.

2. The method of claim 1, wherein forming a motion vector comprises:
detecting an edge of a blood vessel, through which the moving object flows, based on the color Doppler mode image;
setting at least one line perpendicular to the edge;
detecting at least one intersection of the at least one line and the edge;
setting a first area including a predetermined number of pixels to be centered at the at least one intersection;
detecting pixels corresponding to the edge within the first area;
calculating an angle of the edge at the intersection based on the detected pixels; and
forming the motion vector based on the ultrasound data and the angle.

3. The method of claim 2, wherein calculating an angle comprises calculating the angle by using a left-most pixel and a right most pixel among the detected pixels.

4. The method of claim 2, wherein forming the motion vector based on the ultrasound data and the angle comprises:
setting a plurality of second areas on a line segment demarcated by the at least one intersection on the at least one line;
obtaining object-of-interest information for each of the plurality of second areas based at least in part on the ultrasound data for a scanline corresponding to the respective second area;
calculating an average speed in each of the plurality of second areas based on the respectively obtained object-of-interest information;
forming the motion vector for each of the plurality of second areas based on the respective average speed and the angle of the edge.

5. The method of claim 4, wherein the object-of-interest information comprises a speed and direction of movement of the object of interest.

6. The method of claim 1, further comprising displaying the color Doppler mode image with the motion vector set.

7. A system of providing a motion vector on an ultrasound image, comprising:
an image acquisition unit configured to acquire a brightness mode (B-mode) image of a target object;
a region setting unit configured to enable a region of interest (ROI) to be set in the B-mode image, the ROI covering a moving object in the target object;
an ultrasound data acquisition unit configured to acquire ultrasound data for a portion of the target object corresponding to the ROI, the ultrasound data including speed information of the moving object;
an image forming unit configured to form a color Doppler mode image of the portion based on the ultrasound data;
a processor configured to form a motion vector of the moving object based on the ultrasound data and the color Doppler mode image and to set the motion vector on the color Doppler mode image.

8. The system of claim 7, wherein the processor comprises:
an edge detection unit configured to detect an edge of a blood vessel, through which the moving object flows, based on the color Doppler mode image;
a line setting unit configured to set at least one line perpendicular to the edge;
an intersection detection unit configured to detect at least one intersection of the at least one line and the edge;
an area setting unit configured to set a first area including a predetermined number of pixels to be centered at the at least one intersection;
an angle calculation unit configured to detect pixels corresponding to the edge within the first area and to calculate an angle of the edge at the intersection based on the detected pixels; and
a motion vector setting unit configured to form the motion vector based on the ultrasound data and the angle.

9. The system of claim 8, wherein the angle calculation unit is configured to calculate the angle by using a left-most pixel and a right most pixel among the detected pixels.

10. The system of claim 8, wherein the motion vector setting unit is configured to set a plurality of second areas on a line segment demarcated by the at least one intersection on the at least one line, obtain object-of-interest information for each of the plurality of second areas based at least in part on the ultrasound data for a scanline corresponding to the respective second area, calculate an average speed in each of the plurality of second areas based on the respectively obtained object-of-interest information, and form the motion vector for each of the plurality of second areas based on the respective average speed and the angle of the edge.

11. The system of claim 10, wherein the object-of-interest information comprises a speed and direction of movement of the object of interest.

12. The system of claim 7, further comprising a display unit configured to display the color Doppler mode image with the motion vector set.
